(19) United States
(12) Patent Application Publication
(10) Pub. No.: US 2018/0242897 A1
(43) Pub. Date: Aug. 30, 2018

(54) METHOD AND APPARATUS FOR ACCESSING BLOOD FROM AN IV CATHETER INSERTION DEVICE

(71) Applicant: WILLIAM L. SPEARMAN, Beaufort, SC (US)

(72) Inventor: WILLIAM L. SPEARMAN, Beaufort, SC (US)

(21) Appl. No.: 15/444,291

(22) Filed: Feb. 27, 2017

(51) Int. Cl. A61B 5/15; A61M 25/06

(52) U.S. Cl. CPC A61B 5/150992; A61M 25/0693

(57) ABSTRACT

Disclosed is an improved system and method for accessing blood in a flash chamber of an intravenous catheter insertion device. The improved device is mounted on the outside surface of the intravenous catheter insertion device and carries a plunger which moves within the barrel of the device to push on the flash chamber to compress the flash chamber and expel blood through a needle coupled to the flash chamber. Various sizes and/or shapes for the plunger may be provided to accommodate different instances of intravenous catheter insertion devices, and one plunger may be chosen based on a characteristic of the insertion device (e.g., for example, the size or shape of the barrel). This device accommodates other variables in such intravenous catheter insertion devices, such as different kinds of flash plugs (removable/non-removable). The different sizes and/or shapes of the collar may include a collar which has two different configurations, one at either end, and the plunger may be reversed to change the configuration to adapt it to a different intravenous catheter.

### CROSS REFERENCE TO RELATED PATENTS

The present invention is related to my prior patent application Ser. No. 13/340,445 filed Dec. 29, 2011 and entitled “IV Catheter Insertion Device and Method of Using It”, issued as U.S. Pat. No. 8,702,658 on Apr. 22, 2014. This patent is sometimes referred to herein as the “System Patent”.

The present invention is also related to my prior patent application Ser. No. 13/706,324 filed on Dec. 5, 2012 and entitled “Method and System for Accessing Blood from an IV Catheter Insertion Device”, issued as U.S. Pat. No. 8,728,038 on May 20, 2014. This patent is sometimes referred to herein as the “Retrofit Patent”.

The specification and drawings of the System Patent and the Retrofit Patent are hereby incorporated by reference into this patent.

### BACKGROUND OF THE INVENTION

A patient admitted to a hospital, an outpatient treatment center, or an outpatient surgery center will have an intravenous (IV) catheter inserted for easy access for fluids and drugs to be administered to the patient. The IV catheter is typically inserted using an IV catheter insertion device. In addition to placement of an IV catheter, it is frequently necessary for a sample of the patient's blood to be obtained—e.g., for testing blood sugar, blood typing, or other analysis. For many of these blood tests, only a small sample of blood is required. After the IV catheter has been inserted, the healthcare worker may obtain this blood sample by various means. One method would be to have the patient endure another needle stick; either by a needle and syringe to draw an aliquot of blood, or by pricking the patient's finger with a lancet for a few drops of blood.

Several different types of intravenous (IV) catheter insertion devices currently are on the market. One such IV catheter insertion device is marketed by Smiths Medical under its ProtectIV trademark. This device can be seen represented by U.S. Pat. No. 5,000,740. This device uses a passive system to protect the user from needle stick injury. In its design, this device has a flash chamber with a non-removable flash plug filter. Another common intravenous catheter insertion device uses a removable flash plug filter. While several IV catheters include a removable flash plug, a sample of this type of IV catheter would be the Braun Medical safety IV catheter marketed as the Introcan IV catheter. The invention described in this application provides a method and device to allow the user to access the blood within the flash chamber of such devices for testing.

Some have proposed to use some of the blood that remains within IV catheter insertion device after the IV catheter has been inserted in a patient for these simple blood tests. Most IV catheter insertion devices include an integrated flash chamber. Red blood appears within the flash chamber of an IV catheter insertion device as the operator inserts the needle of the IV catheter insertion device into the vein to show that the IV catheter has entered a vein. The operator watches the flash chamber continuing to fill with blood as the operator advances the IV catheter into the vein, thereby ensuring the IV catheter remains properly positioned within the vein. Usually, the proximal end of the flash chamber is blocked by a flash plug. The flash plug (or flash plug filter) typically includes a filter material that allows air to vent from the flash chamber as the blood or fluid fills the chamber, but prevents the blood or fluid from passing from the flash chamber. IV catheter insertion devices of the prior art may have one of two different types of flash plugs. One type of IV catheter insertion device, such as described in U.S. Pat. No. 5,000,740, has a fixed or non-removable flash plug. Another type of prior art IV catheter insertion device is seen in U.S. Pat. No. 4,917,671; this type of IV insertion device has a removable flash plug at the end of its flash chamber.

When an IV catheter has been placed within the vein, the remainder of the IV catheter insertion device is removed from the patient. A small amount of the patient's blood remains within the flash chamber of the IV catheter insertion device. By accessing the patient's blood from the flash chamber of the IV catheter insertion device, the operator may be able to avoid an additional needle stick to the patient. There are some known approaches to access blood within the flash chamber of an IV catheter insertion device. One IV catheter insertion device, such as U.S. Pat. No. 5,000,740 which is currently marketed by Smiths Medical under its ProtectIV trademark, offers a unique problem when trying to access the blood within the flash chamber since it has a non-removable flash plug. As such, users must adapt special methods if they wish to access the blood within the flash chamber. One such method to access this blood in an IV insertion device with a non-removable flash plug involves finding and using an external probe, such as a pen or a golf tee, to push the internal and non-removable flash plug within the flash chamber to expel blood from the flash chamber. The use of an external device such as a golf tee or pen is undesirable for several reasons. The external device must be stored some place that is easily accessible to the operator when the operator is ready to use it. The use of an external device is inefficient as the worker must look for or gather additional equipment when accessing the captured blood or fluid and replace the additional equipment for its next use, if the worker were to reuse any external probe or device (including a pen or a golf tee), cross-contamination may occur from one flash chamber to another, putting other patients and the healthcare worker at risk. In addition, the user must align the external probe with the flash plug while pushing the device and flash plug forward within the flash chamber while at the same time keeping the tip of the needle over the correct spot on the testing strip. A recent device and system by Spearman, taught in my U.S. Pat. Nos. 8,728,038 and 8,702,658, provides the user a device to access this blood simply and efficiently. However, the device described in U.S. Pat. No. 8,728,038 and U.S. Pat. No. 8,702,658 must be either included by the manufacturer during assembly of the IV catheter device or attached by the end user. The device attached by the end user works well, but because of its small size may, at times, be difficult for the user to handle and/or insert. The present invention is easier to handle and insert.

In IV catheter insertion devices that have removable flash plugs, another prior art approach to access the blood in a flash chamber requires the healthcare worker to remove the flash plug, thereby opening the flash chamber to access the blood within the flash chamber for testing. This action may expose a healthcare worker needlessly to hazardous material (such as blood or other bodily fluid) that may spill from the flash chamber. In many instances, even opening the flash chamber by removing the flash plug may not make access to the blood easy. The operator may need to tap the flash chamber against a solid surface to get the blood to drop onto the testing strip. The present invention reduces the chance of blood exposure by sealing the flash chamber after the flash plug is removed and replaced by the invention. The invention then provides a means to express the blood from the flash chamber in a safe and controlled manner.

While various prior art methods may allow the healthcare worker to access the blood or fluid within the flash chamber of an IV catheter insertion device, these methods present an inefficient solution with undesirable aspects and limitations.

My System Patent (U.S. Pat. No. 8,702,658) provides a design of a complete IV insertion device in which blood can be accessed from the device. This design disclosed in this patent (U.S. Pat. No. 8,702,658) requires the device be included in the manufacture and assembly of the IV catheter insertion device. However, many institutions and professionals continue to use IV catheter insertion devices of the prior design, and it is desired to provide a way for them to access the blood which is located within the flash chamber for testing purposes.

The present invention addresses some of these undesirable limitations and aspects to provide an improved device and method for accessing blood from an IV catheter insertion device without the use of undesirable additional hardware and without unnecessary risks.

This new method and device will thereby increase the efficiency of the healthcare worker and reduce the hazardous exposure of bodily fluids to the workers and patients. Other disadvantages and limitation of the prior art systems will be apparent to those skilled in the art.

### SUMMARY OF THE INVENTION

The present invention provides a system and method for accessing blood within the flash chamber of IV insertion devices that have both non-removable and removable flash plug filters. The intravenous insertion devices with which this invention will work include devices with a non-removable flash plug, such as the device sold under the ProtectIV trademark (and similar devices), and devices with removable flash plug filters, similar to the Braun Medical IV catheter sold under the Introcan trademark and the Jelco IntuitIV trademark catheter (and similar devices). The invention allows the user to access the blood within the flash chambers of these intravenous catheter devices in a controlled manner upon demand with greater ease and less risk to the operator. The invention permits the operator to deliver this blood sample to a testing strip or device for point of care testing.

The present invention has the advantage of reducing the number of needle sticks to a patient by using blood or bodily fluid that has been collected within the IV catheter insertion device when the IV catheter is placed. That is, the fluid sample used for testing is a by-product of the use of the IV catheter insertion device.

Applicant teaches in this patent application that blood collected within the flash chamber of an intravenous catheter insertion device with a non-removable flash plug can be accessed with an attachable device which becomes attached to the IV catheter insertion device and allows access to blood within the flash chamber safely and efficiently. The blood or fluid thus collected can be used for analysis or testing without requiring the healthcare worker to inflict additional needle sticks to a patient.

It is desirable that any part of an IV catheter insertion device be simple to make. It is also desirable that the device be self-contained and not require additional tools, e.g., an external device like a golf tee or pen, to access the blood contained in the flash chamber.

It is also desirable that when the operator must open the flash chamber of an IV catheter insertion device to access the blood therein that the device easily allow the operator to express a blood sample from the flash chamber onto a point of care testing strip while minimizing blood exposure to the operator. As the invention is attached to the open flash chamber, it seals the proximal end of the flash chamber and reduces the chance of blood exposure to the user.

It is further desirable that the device can be easily attached to the IV insertion device after the IV catheter has been inserted into the patient's vein with minimal teaching to the operator.

The present invention overcomes the disadvantages and limitations of the prior art systems while providing a simple, yet effective, way of accessing the blood contained in the flash chamber of an IV catheter insertion device for testing and analysis without need for a golf tee or ballpoint pen.

The present invention provides a way of using an IV catheter insertion device of the prior art while being able to access blood which is located within the flash chamber for testing.

The present invention has the advantage that it works both with intravenous catheter insertion devices with flash plugs which are not removable (for example, as described in U.S. Pat. No. 5,000,740) and with intravenous catheter insertion devices with removable flash plug filters. Because the invention works with both types of IV catheter insertion devices, the costs of manufacture and assembly of the invention is reduced from the prior art described by my U.S. Pat. No. 8,728,038 and U.S. Pat. No. 8,702,658 referenced above.

The present invention overcomes some of the limitations and disadvantages of prior art systems.

Other objects and advantages of the present invention will be apparent to those of ordinary skill in the art to which the present invention applies.

### BRIEF DESCRIPTION OF DRAWINGS

FIG. 1 illustrates samples of IV catheter insertion devices, top version with non-removable flash plug filter and bottom, sample IV catheter with removable flash plug.

FIG. 2A shows one collar of present invention and FIG. 2B shows a cross section view of collar of FIG. 2A.

FIG. 3A showing one type of plunger for the present invention (usable with an IV catheter insertion device with a non-removable flash plug) and FIG. 3B showing an alternate design for a plunger (usable with an IV catheter insertion device with a removable flash plug filter).

FIGS. 4A, 4B and 4C show a plunger and collar assembled, with FIG. 4B showing a cross sectional view of the plunger and collar assembled.

FIG. 5A shows the assembly of the plunger and the collar and FIG. 5B shows one version of an IV catheter insertion device with non-removable flash plug after IV insertion, with blood within its flash chamber.

FIG. 6A shows a cross sectional view of invention inserted into proximal end of IV catheter device (with the plunger retracted) and FIG. 6B shows a cross sectional view with its plunger advanced showing the filter expressing blood from the flash chamber through the needle.

FIG. 7A shows the collar and plunger of the present invention assembled before insertion in an Intravenous insertion device with a removable flash plug (in FIG. 7B).

FIG. 8A illustrates a cross sectional of inventive plunger and collar attached to the proximal end of the flash chamber of an IV catheter insertion device with flash plug removed, with the plunger retracted). The length of the plunger is somewhat a matter of design choice and may depend on the size of the IV catheter insertion device to which the plunger is being attached. FIG. 8B is a cross sectional view of the collar and plunger attached to IV catheter insertion device with its plunger depressed to express blood from flash chamber through the needle.

FIG. 9A is a cross sectional view of an IV catheter with its flash plug removed and the collar and plunger of the present invention attached. FIG. 9B is a cross sectional view of the IV catheter insertion device of FIG. 9A with the plunger advanced into the flash chamber with a plug forcing blood distally within the flash chamber.

### DETAILED DESCRIPTION OF THE PREFERRED EMBODIMENTS

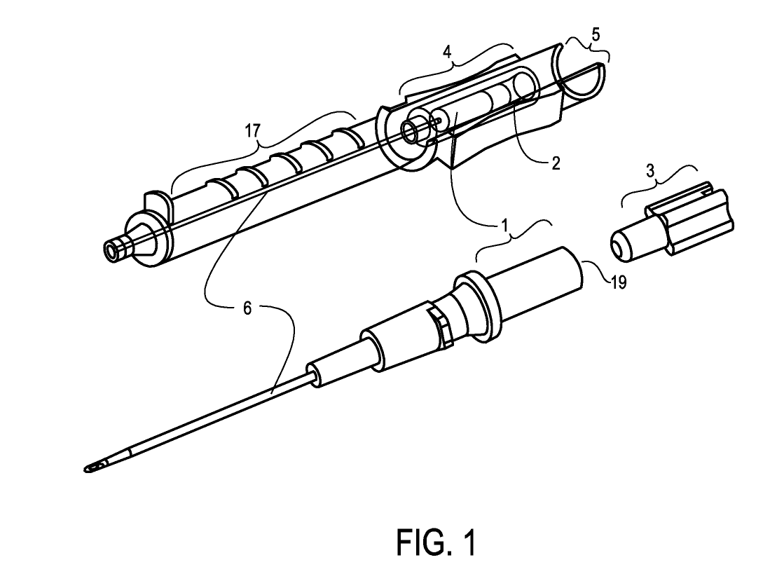

FIG. 1 shows two different IV catheter insertion assemblies or devices, one with a non-removable flash plug filter 2 and the other with a removable flash plug 3. The top view shows the assembly after placement of the IV catheter with a safety tube 17 advanced over a needle 6.

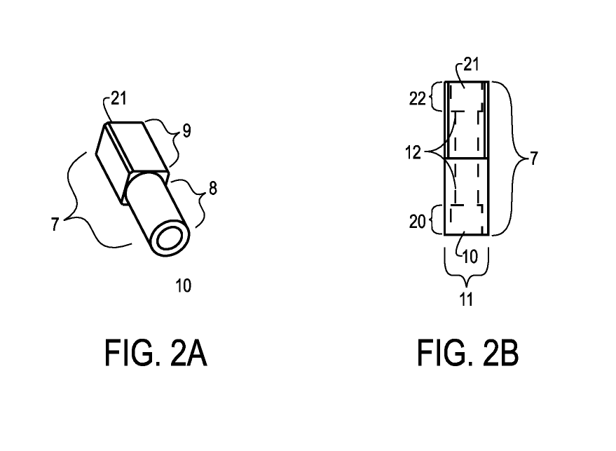

FIG. 2A illustrates a perspective view of one form of collar 7 useful in the present invention.

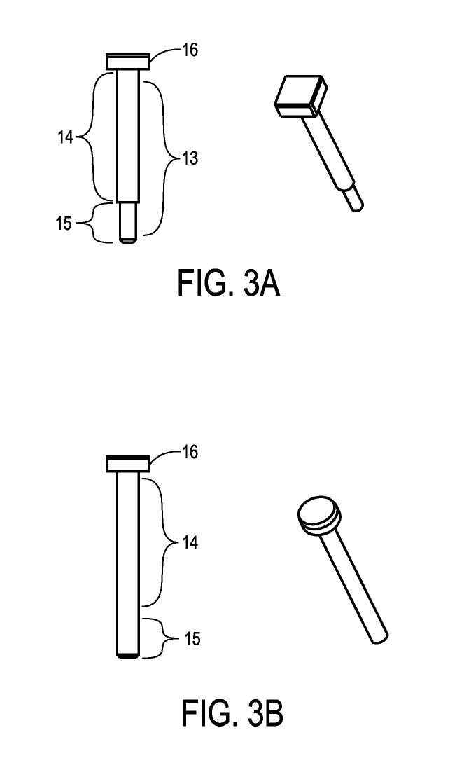

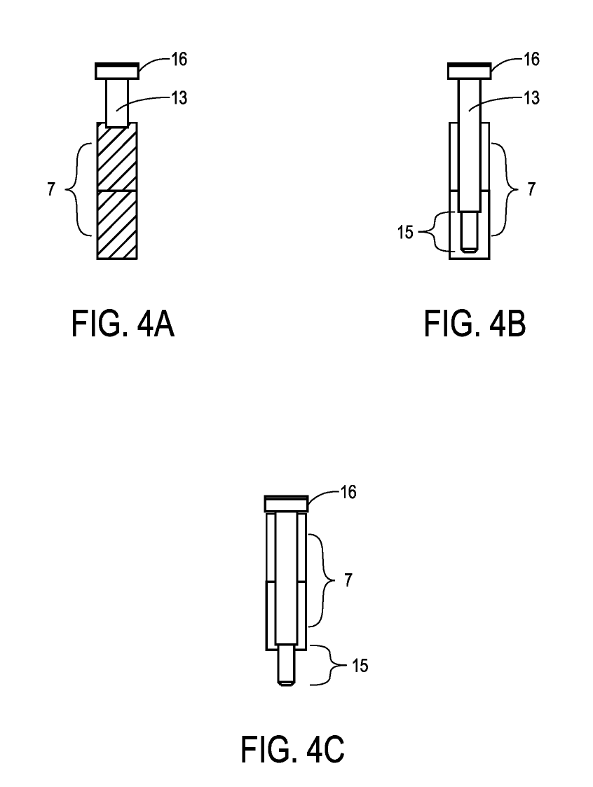

The invention comprises two parts assembled together. One part is a collar 7 (seen in FIGS. 2A and 2B) which serves to secure the assembly to the barrel 5 of the IV catheter device (of FIG. 1). The collar 7 has a central or internal channel 12. The distal end 8 of the collar 7 is cylindrical. The proximal end 9 of the collar 7 may be of any shape, but herein is illustrated as squared. The other piece of the invention is a movable member or plunger 13 (as shown in FIGS. 3A and 3B). The shaft 14 of the plunger is cylindrical and sized to match (or fit within while allowing movement) the central channel 12 of the collar 7. The distal end 15 of the plunger shaft 14 may be of different diameters depending on the type of IV catheter insertion device for which it is provided. When the invention is assembled (see FIG. 4A, 4B), the plunger 13 sits within the central channel 12 of the collar 7. Initially, the plunger 13 is in a first or retracted position (shown in FIG. 4A). By depressing a plunger cap 16, the plunger 13 is moved to a second or forward position 2 (shown in FIG. 4C). In position 2 (FIG. 4C), the distal tip 15 extends beyond the distal collar 7.

While the invention can be used with both types of IV catheter insertion devices (IV devices with a non-removable flash plug (for example FIG. 1, top illustration) and IV devices with removable flash plugs, for example (FIG. 1, bottom illustration)), the use of the invention differs slightly depending on the type of IV catheter device.

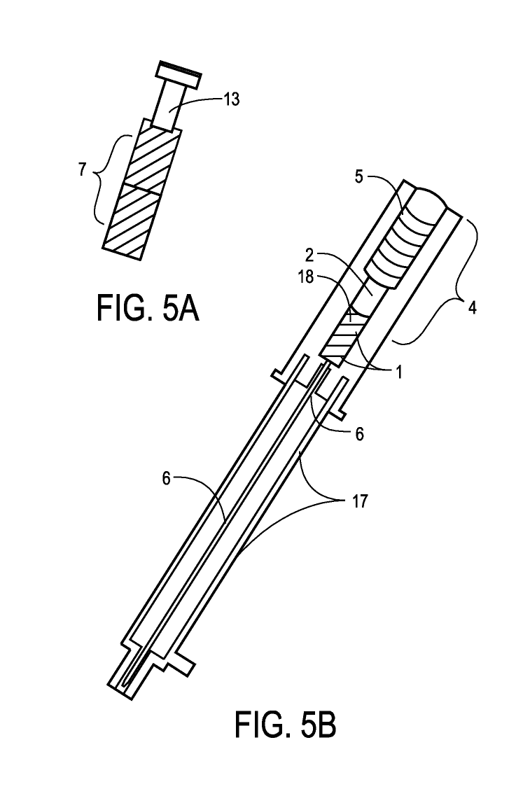

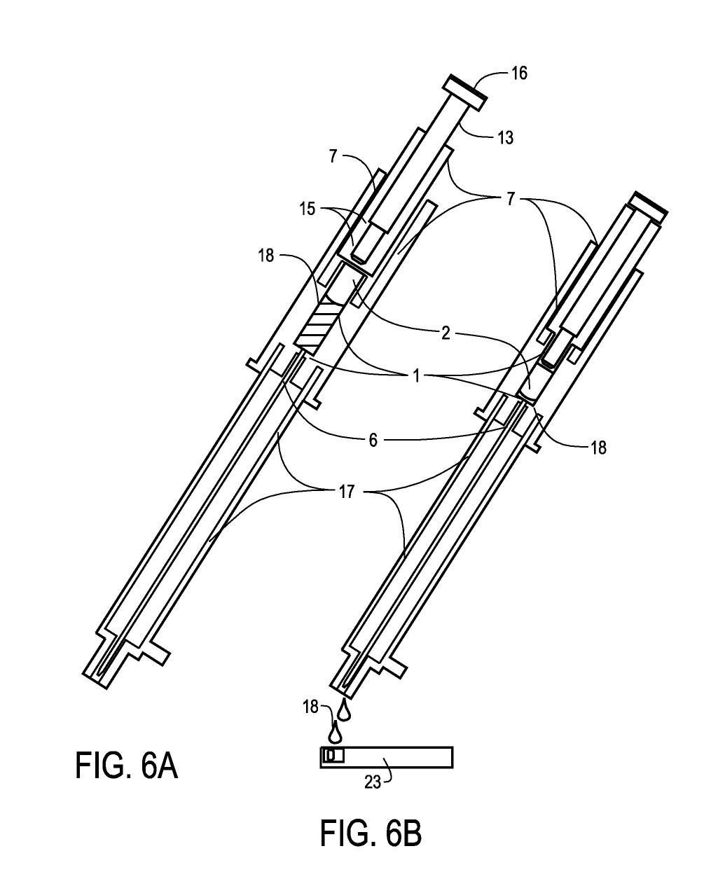

In one preferred form, when used with an IV catheter device with a non-removable flash plug, the invention is used as follows: (refer to FIG. 5 and FIG. 6). After the IV catheter has been placed, blood 18 will be found in a flash chamber 1 of the IV catheter device. As the IV catheter was inserted, the protective shield/tube 17 slides forward to cover the needle 6. Finger grips 4 form a channel or tubular body 5 that surrounds and frames a flash plug filter 2. The external diameter of the distal collar 8 fits snugly within the channel or tubular body 5 framed by the finger grips or guards 4. The user inserts the cylindrical distal tip 8 of the collar 7. As the distal tip 8 is generally cylindrical like the channel or tubular body 5 formed by the finger grips 4, the collar 7 may be inserted in any orientation. The user advances the collar 7 until it stops against the distal end of the channel 5 formed by the finger grips 4. In this position (FIG. 6) the distal opening 10 of the collar 7 surrounds the proximal end of the flash chamber 2 (FIG. 6A). In this position (FIG. 6A), the collar aligns the distal tip 15 of the plunger 13 within the central channel 12 of the collar 7 with the proximal end of the flash plug filter 2. The user can then position the distal tip of the needle 6 over the blood receptacle, a testing strip 23. (FIG. 6B) The user then advances the plunger 13 by pushing on the plunger top 16. As the plunger 13 is moved from its first or retracted position (position 1) to its advanced position (position 2) (FIG. 6A), the advancing plunger tip 15 forces the flash plug filter 2 distally within the flash chamber 1. As the flash plug filter 2 advances distally, the blood 18 within the flash chamber 1 is forced to move distally through the needle 6 and onto the testing strip 23. For this type of IV catheter insertion device, the distal tip 15 of the plunger 13 is a size that fits within the flash chamber 1 of that IV catheter device. The shaft 14 of the plunger fits the central channel 12 of the collar 7 regardless of type of IV catheter device. After the blood 18 has been expressed onto the testing strip 23, the entire IV catheter device with the invention attached (FIG. 6B) may be discarded.

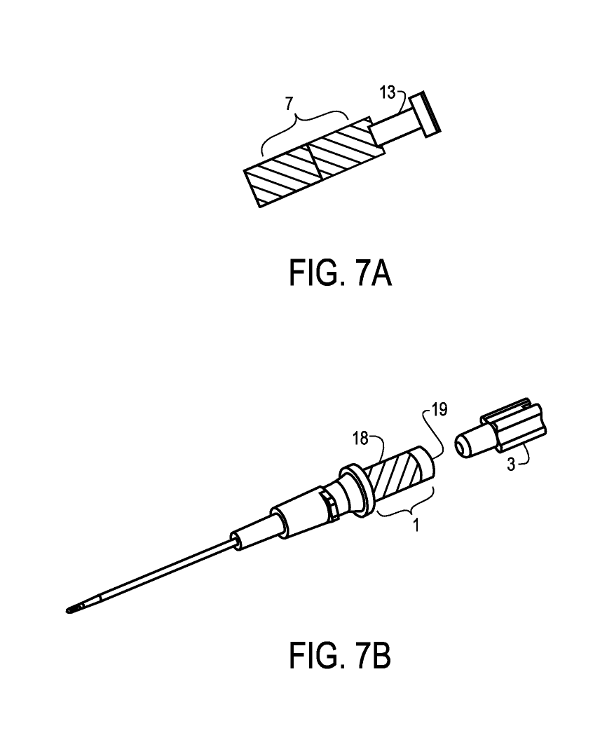

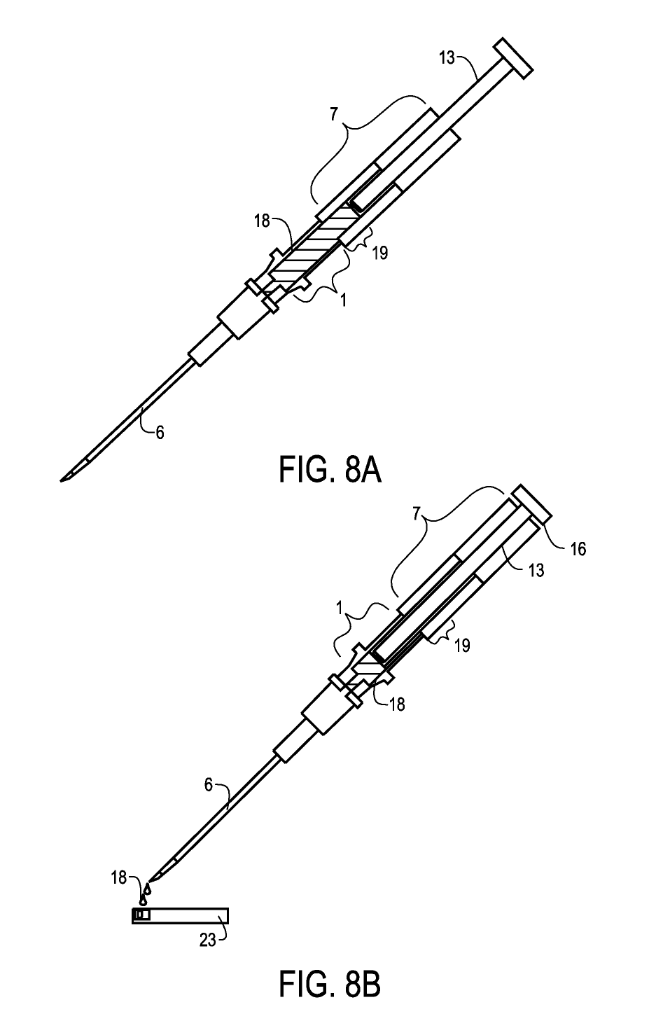

In another preferred form, the invention can be used with IV catheter insertion devices with removable flash plugs 3 as shown in example in FIG. 18 and FIGS. 7 and 8. After the operator has placed the IV catheter, the IV catheter (FIG. 7B) has blood 18 within the flash chamber 1. In this illustration, the flash plug filter 3 has been removed. The operator then attaches the invention (FIG. 7A) by sliding the distal end 20 of the collar 7 over the proximal end 19 of the flash chamber 1 after the flash plug 3 has been removed. (FIG. 8). The internal diameter of the distal opening 10 of the collar 7 fits snugly over the external diameter of the proximal flash chamber 19. The fit of the collar 7 onto the proximal end 19 of the flash chamber 1 is secured by friction and prevents or reduces the backflow of blood 18 from the flash chamber. The operator can then move the plunger 13 from position 1 to position 2 by depressing the plunger cap 16. As the plunger tip 15 advances within the flash chamber 1, the outer diameter of the plunger tip 15 fills the inner diameter of the flash chamber 1 and therefore forces the blood 18 within the flash chamber 1 out of the flash chamber through the needle 6 and onto the testing strip 23.

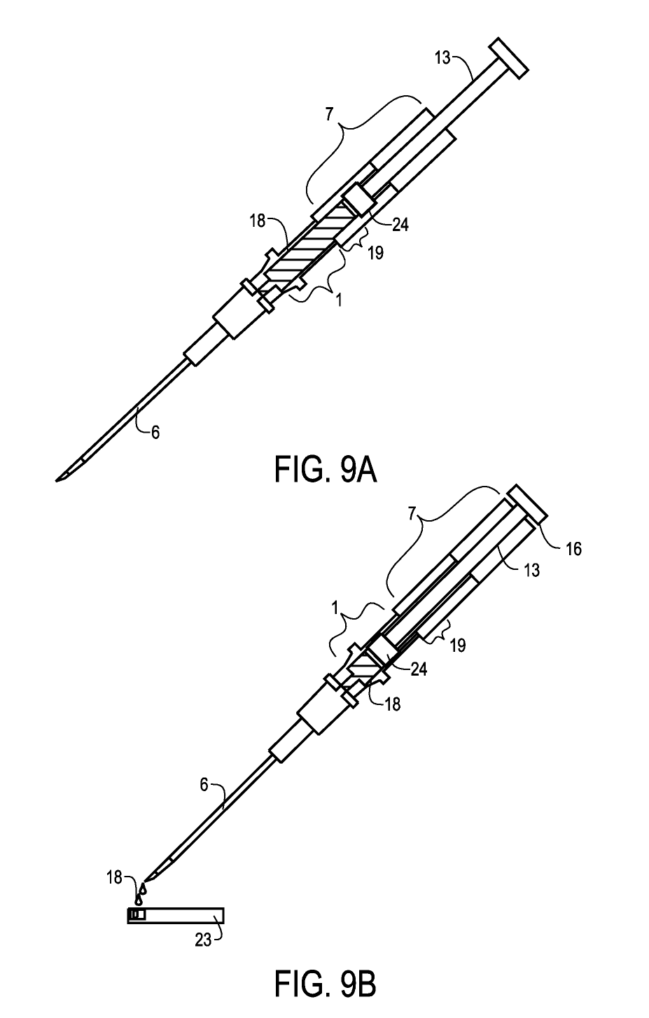

In an alternate form (FIG. 9), when used with an IV catheter device with a removable flash plug, the invention would be as above but with the addition of a plug 24 within the central channel 12 of the collar 7 and distal to the plunger tip 15. The plug may be composed of expandable and/or compressible material that may or not be of filter type material. The material may allow air to escape but will repel the blood within the flash chamber. In practice, after placement of an IV catheter, the operator would remove the removable flash plug 3 and attach the collar of the invention 7 to the proximal end of the open flash chamber 19 (FIG. 9A) as described in the above paragraph. Once the needle 6 tip is positioned over the testing strip 23, the operator depresses the plunger 16. (FIG. 9B) As the plunger shaft 13 is advanced, the plunger tip 15 forces the plug 24 distally into the flash chamber 1. As the plug 24 advances distally within the flash chamber 1, it pushes the blood 18 distally within the flash chamber 1 and out of the needle 6 onto the testing strip 23.

The design of the invention reduces the number of pieces needed for different types of IV catheter devices produced by different manufacturers. The central channel 12 of the collar 7 is a standard size and the outer diameter 11 of the of the distal tip of collar 8 is standard to the inner diameter of the channel 5 formed by the finger grips 4 of the non-removable IV catheter insertion device (e.g., FIG. 1A). The outer diameter of the flash chambers 1 with removable flash plugs of different IV catheter devices (from different manufacturers) may vary. The inner diameter of the flash chambers 1 of IV insertion devices with removable flash chambers are generally a standard size. Despite the variables, the manufacturer of this invention may fit different devices to different IV catheter devices by simply changing the size of the distal tip 15 of the plunger 13 and the internal diameter 10 distal end 20 of the collar 7. As such, in another preferred form, the invention could be manufactured such that the collar 7 has a central channel 12 that fits the mid-shaft of the plunger 14. One end of the collar 20 can have one sized internal diameter 10 while the other end 22 could have a different internal diameter 21. The internal diameter of each end could correspond to the outer diameter of the flash chambers of IV catheters with removable flash plugs produced by different manufacturers. As the flash chambers of these IV catheter devices with removable flash plugs have different outer diameters but standard inner diameters, the plunger could be the same for these IV devices produced by different manufacturers. Therefore, the manufacturer could build the invention to fit three different IV catheter devices with one collar and two different plungers. For example, if the manufacturer would like to sell the device to three hospitals that all use IV catheter devices from different manufactures. Hospital A uses an IV device with a non-removable flash plug (such as a ProtectIV marketed by Smiths Medical). Hospital B uses one type of IV device with a removable flash plug (such as an Introcan IV marketed by Braun Medical). And, Hospital C uses a different IV catheter with a removable flash plug (such as the Acuvance IV market by Jelco/Smiths Medical). Hospital A's IV catheter is totally different from the IV catheter of Hospital B and C. And, the outer diameter of the flash chambers of the IV catheters of Hospital B and C are different. Typically, the manufacturer of this device would need to build and use six different manufacturing molds, that is, one collar and one plunger for each of the three different IV catheters. However, with this invention, the manufacturer can now mold one collar with different internal diameters on the ends of the collar (FIG. 2B) (10, 21). As the flash chambers of the IV devices from Hospital B and C have standard (similar) internal diameters. Therefore, only one plunger need be manufactured that will fit the internal diameters of the flash chambers of those IV catheters with removable flash plugs. As such, the manufacturer of the invention can use one mold for the collar, one mold for the plunger that fits IV catheters with non-removable flash plugs (FIG. 3A), and another mold to produce a plunger that fits IV devices with removable flash plugs (FIG. 3B). So rather than six molds, the manufacturer can produce devices that fit three different IV devices with only 3 molds. The manufacturer need only assemble the correct plunger with the correct orientation of the collar to service the different IV catheters. Alternatively, the manufacturer could use one plunger but add a plug of varying size to fit the internal diameter of the flash chamber of the specific IV catheter insertion device.

Of course, many modifications of the preferred embodiment can be made without departing from the spirit of the present invention. Furthermore, one of ordinary skill in the relevant art will realize that some features can be used to advantage without the corresponding use of other features. For example, the use of a collar or plunger attaching member which fits completely around the barrel is described with some particularity, but the present invention can be accomplished in other ways without departing from the principles of the present invention. As an example, the support member may only partially surround the barrel and may be attached by some other known method, Accordingly, the present description should be considered as merely illustrative of the principles of the present invention and not in limitation thereof. Other modifications and adaptations can be made, if desired, while achieving a similar objective.

Having thus described the invention, what is claimed is:

1. A method of accessing blood held within a flash chamber of an intravenous catheter insertion device having a barrel surrounding the flash chamber and a needle communicating with the flash chamber, the steps of the method comprising:
attaching a collar with at least a portion extending outside of the barrel surrounding the flash chamber;
mounting a plunger to the collar, the plunger movable within the barrel; and
moving the plunger from a first position with respect to the collar and the barrel where the plunger does not press on the flash chamber to a second position pressing on the flash chamber, where the pressing on the flash chamber causes blood from within the flash chamber to be directed out the needle for testing the blood.

2. The steps of the method of claim 1 further including the steps of determining at least one characteristic of the intravenous catheter insertion device; using that characteristic to select a collar to use; and
mounting the selected collar to the barrel to allow the plunger to move with respect to the barrel and the flash chamber to expel blood from the flash chamber in response to the movement of the plunger.

3. An apparatus for accessing blood in a flash chamber of an intravenous catheter insertion device comprising:
a collar with an attachment system for attaching to a barrel of an intravenous catheter insertion device, the collar including a portion extending outside of the barrel;
a plunger mounted to the collar and positioned to be located within the barrel of the device;
the plunger including a handle for moving the plunger from a first position not compressing the flash chamber to a second position compressing the flash chamber, whereby the movement of the handle compresses the flash chamber and expels blood from the flash chamber through the needle for testing.

4. An apparatus for accessing the blood of the type described in claim 3 wherein a plurality of different plunger and collar assemblies are provided and one plunger and collar assembly is chosen based on the structure of intravenous catheter insertion device.

5. An apparatus of the type described in claim 4 wherein a single plunger and collar assembly has a first configuration at one end and a second configuration at its other end, and the user determines which configuration of the plunger and collar assembly to position adjacent the flash chamber based on a characteristic of the intravenous catheter insertion device.

6. An apparatus of the type described in claim 5 wherein the characteristic is the shape of the barrel.

7. An apparatus of the type described in claim 5 wherein the characteristic is the bore of the barrel.

8. An apparatus of the type described in claim 5 wherein the characteristic is the type of flash plug mounted within the insertion device.

9. An apparatus of the type described in claim 5 wherein the characteristic is the is the geometry of the flash plug.

10. A method of accessing blood held within a flash chamber of an intravenous catheter insertion device having a barrel surrounding the flash chamber and a needle communicating with the flash chamber, the steps of the method comprising:
attaching a support member to the barrel surrounding the flash chamber with a portion extending outside the barrel;
mounting a plunger to the support member, the plunger movable within the barrel; and
moving the plunger from a first position with respect to the support member and the barrel where the plunger does not press on the flash chamber to a second position pressing on the flash chamber, where the pressing on the flash chamber causes blood from within the flash chamber to be directed out the needle for testing the blood.

11. The steps of the method of claim 10 further including the steps of determining at least one characteristic of the intravenous catheter insertion device;
using that characteristic to select a plunger and support member combination to use;
and attaching the selected plunger and support member to the barrel to allow the plunger to move with respect to the support member and the flash chamber to expel blood from the flash chamber in response to the movement of the plunger.

12. An apparatus for accessing blood within a flash chamber of an intravenous catheter insertion device having a barrel surrounding the flash chamber where the flash chamber is attached to a needle, the apparatus comprising:
an attaching member secured to the barrel with at least a portion of the attaching member extending outside of the barrel; and
a plunger movably attached to the attaching member for movement between a first position with respect to the barrel in which the flash chamber is not compressed by the plunger and a second position in which the flash chamber is compressed by the plunger, whereby when the plunger is moved from the first position to the second position, blood in the flash chamber is expelled from the flash chamber through the needle.

13. The apparatus of claim 12 wherein a plurality of plunger designs are provided and the user selects one plunger design based on a characteristic of the barrel to which the apparatus is being attached.

14. The apparatus of claim 13 wherein a single collar has one design at one end and a different design at the other end and the user selects which end of the collar to position adjacent the flash chamber based on the characteristic of the barrel.
